(19) United States
(12) Patent Application Publication
(10) Pub. No.: US 2010/0170043 A1
(43) Pub. Date: Jul. 8, 2010

(54) APPARATUS FOR MONITORING VITAL SIGNS

(75) Inventors: Steven J. Young, Los Gatos, CA (US); Richard V. Rifredi, Los Gatos, CA (US); Yuri Zhovnirovsky, Albany, CA (US)

Correspondence Address: YOUNG BASILE, 3001 WEST BIG BEAVER ROAD, SUITE 624, TROY, MI, 48084, US

(73) Assignee: BAM LABS, INC., San Jose, CA (US)

(21) Appl. No.: 12/349,167

(22) Filed: Jan. 6, 2009

(51) Int. Cl. A47C 27/08; A61B 5/0205; A61B 5/08

(52) U.S. Cl. 5/706; 600/484; 600/534

(57) ABSTRACT

A sleep monitoring system includes a fluid bladder. A pump is in fluid communication with the fluid bladder, and the pump is operable to increase a fluid pressure within the fluid bladder. A sensor is packaged with the pump, and the sensor is in fluid communication with the fluid bladder and is operative to determine a pressure within the fluid bladder. A controller is configured to determine the at least one vital sign based on the pressure within the fluid bladder.

### CROSS-REFERENCE TO RELATED APPLICATION

This application claims priority from pending U.S. patent application Ser. No. 11/849,051, filed Aug. 31, 2007, and U.S. Provisional Application Ser. No. 60/846,642 filed Sep. 22, 2006, each of which is incorporated herein in its entirety by reference.

### FIELD OF THE. INVENTION

The present invention pertains to a vital sign monitoring apparatus.

### BACKGROUND

Historically, monitoring vital signs of a person has required expensive equipment, such as an electrocardiogram (EKG) or a ballistocardiograph (BCG). In addition to being prohibitively expensive for many situations (e.g., home use), both EKGs and BCGs can be too cumbersome for use outside of medical facilities. EKGs, for example, typically necessitate attaching electrodes to the bodies of users, while BCGs rely on large, heavy, and unaesthetic force-measuring platforms that users lie on.

In more recent times, devices including piezoelectric films or arrays of sensors have been developed to measure heart and respiration rates. A user can lie on the device, and the film or sensors can generate a signal indicate of the user's heart rate and/or respiration rate. However, these devices can also be expensive.

### SUMMARY

Some known air mattresses each include a pump connected to the respective air mattress by a hose. The pump can produce a high pressure to force air into the air mattress. However, the air mattress can lose air over time, causing the pressure in the air mattress to drop beneath a preset level. In order to reduce the problems associated with air loss, the pump can include a pressure sensor, and the pump automatically turn on when the pressure drops below the preset level. As a result, a user does not have to periodically turn on the pump to increase the air pressure in the air mattress.

A pressure sensor used to communicate with the pump can additionally be leveraged to detect vital signs, such as a heart rate and respiratory rate of a person lying on the air mattress. According to an example of a sleep monitoring system that can determine at least one vital sign of a person, the sleep monitoring system includes a fluid bladder. A pump is in fluid communication with the fluid bladder, and the pump is operable to increase a fluid pressure within the fluid bladder. A sensor is packaged with the pump. The sensor is in fluid communication with the fluid bladder, and the sensor is operative to determine a pressure within the fluid bladder. A controller is configured to determine the at least one vital sign based on the pressure within the fluid bladder.

As a result, the cost of the sleep monitoring system can be reduced compared to many vital sign monitoring devices. Further, since the sleep monitoring system can be less cumbersome to use compared to many vital sign monitoring devices, the sleep monitoring systems can be used outside of a medical center environment. Additionally, since a pump of a conventional air mattress may include a pressure sensor, that pressure sensor can be leveraged to create the sleep monitoring system by merely providing a software upgrade. Also, by analyzing sleep information generated over time, the sleep monitoring system can provide a pressure setting customized for a specific user to improve the user's sleep.

Another example of sleep monitoring system is also provided. The sleep monitoring system includes a fluid bladder. A pump is spaced from the fluid bladder, and the pump has a housing containing pump components and defining a fluid inlet for receiving fluid and a fluid outlet for outputting fluid pressurized by the pump. An elongate conduit fluidly couples the fluid outlet of the pump and the fluid bladder. The conduit provides a passage for the fluid pressurized by the pump to increase a fluid pressure within the fluid bladder. A pressure sensor is physically coupled to an interior of the pump housing such that the sensor is part of an integral pump unit, and the pressure sensor is configured to detect a pressure of fluid at the fluid outlet of the pump. A controller is configured to determine the at least one vital sign based on the pressure within the fluid bladder.

An example of sleeping pad is also provided. The pad includes a fluid bladder. A pressure sensor is in fluid communication with the fluid bladder and is operable to detect a fluid pressure within the fluid bladder. A controller is in communication with the pressure sensor and is operable to determine at least one vital sign of a user based on the pressure within the fluid bladder. A memory for storing historical data including the pressure within the fluid bladder and the at least one vital sign and a processor for determining a sleep quality correlation between the pressure within the fluid bladder and the at least one vital sign based on the historical data are also included. A pressurized fluid source is operable to increase a pressure within the fluid bladder when the sleep quality correlation indicates a sleep quality of the user would improve if the pressure within the fluid bladder were higher

### BRIEF DESCRIPTION OF THE DRAWINGS

The description herein makes reference to the accompanying drawings, wherein like reference numerals refer to like parts throughout the several views, and wherein:

FIG. 1 is an end view of a sleep monitoring system including an air mattress and a pump;

FIG. 2 is a schematic view of the sleep monitoring system of FIG. 1;

FIG. 3 is a cross-section view of the air mattress of FIG. 1 along line A-A in FIG. 1; and

FIG. 4 is a flowchart showing a determination of a pressure setting.

### DETAILED DESCRIPTION

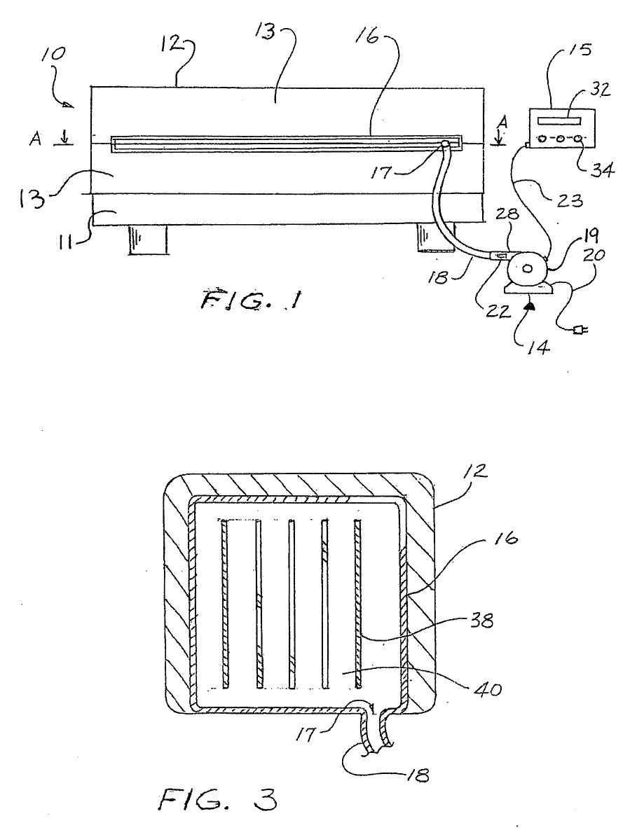

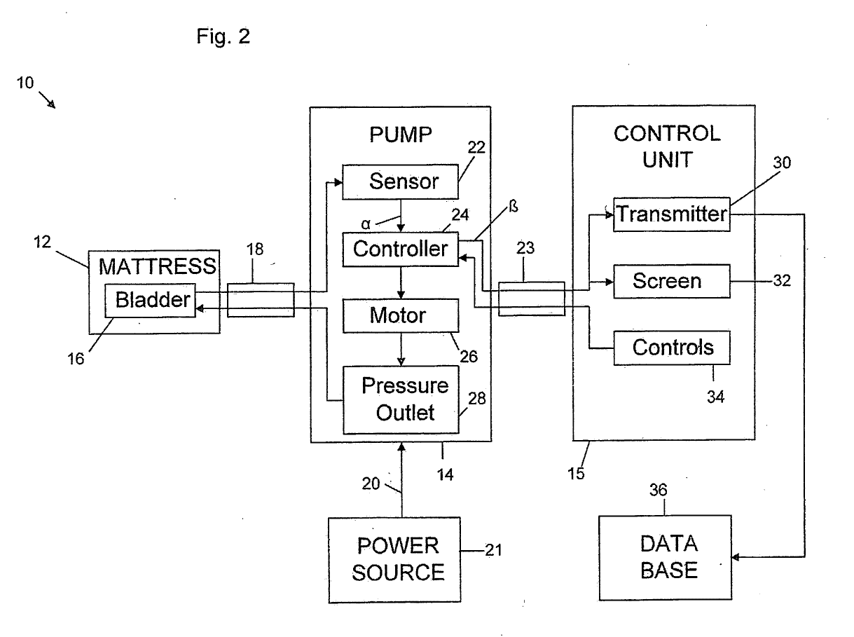

A sleep monitoring system 10 can include a mattress 12, a pump 14, and a control unit 15 as shown in FIGS. 1 and 2. The mattress 12 can include a fluid bladder 16. The mattress 12 can be sized for use on a king-size, queen-size, full, twin, or other sized bed frame 11. The mattress 12 can additionally include a padding layer 13 on top of and/or beneath the fluid bladder 16 as shown in FIG. 1. The padding layers 13 can include one or more of a foam pad, a box spring, an additional fluid bladder, a straw-filled pad, a feather-filled pad, a sawdust-filled pad, a spring-based pad, and/or another type of padding that offers flexibility and/or softness. Alternatively, the mattress 12 can be used sized for use in a chair, hospital bed, crib, or another structure for which padding can add comfort.

The bladder 16 can hold air or another fluid, such as water. In addition to holding air or another fluid, the bladder 16 can enclose foam or another material through which fluid waves of an expected magnitude can propagate a sufficient distance without being too dampened. The fluid bladder 16 can be sized to have a surface area nearly as large as a surface area of a top side of the mattress 12 to allow the detection of a user's vital signs regardless of the position of the user. Alternatively, the bladder 16 can have a smaller size, such as a size covering an area of the mattress 12 above which the user's heart and/or lungs are expected to be positioned (e.g., a one foot by one foot square for an adult user). Even if the user is positioned on the mattress 12 such that the user's heart and/or lungs are not directly above the bladder 16, pressure fluctuations caused by the user may still be received by the bladder 16. The pressure in the fluid bladder 16 can vary depending on the amount of fluid in the bladder 16, whether a user is lying on the bladder 16, the heart rate of a user lying on the bladder 16, the respiration rate of a user lying on the bladder 16, other movement of a user lying on the bladder 16 (e.g., rolling or limb movement), the temperature of the fluid in the bladder 16, and other considerations.

The pump 14 can be a separate unit from the mattress 12 and can be fluidly coupled to an air inlet 17 of the bladder 16 via a hose 18 as shown in FIGS. 1 and 2. However, the pump 14 can alternatively be integral with the mattress 12 such that the pump 14 can output high pressure fluid directly into the bladder 16 instead of through the hose 18. The pump 14 can be a rotary type pump or another type of pump. The pump 14 can include an electric line 20 for connection to an outlet 21 as shown in FIG. 2 or for connection to another power source, and the pump 14 can also include a data line 23 for communication with the control unit 15. Alternatively, the pump 14 can include a self-contained power source, such as one or more batteries.

As shown in FIG. 2, the pump 14 can be packaged with a sensor 22 and a controller 24 in communication with both the sensor 22 and the control unit 15. That is, the pump 14 and sensor 22 can be part of an integral unit. For example, a pump housing 19 that acts as a casing containing components of the pump 14 can also contain the sensor 22. The pump housing 19 can be made from a rigid material (e.g., ABS plastic, polypropylene, a metal, or another material), and the pump housing 19 in its assembled form containing components of the pump 14 and sensor 22 can have the appearance of a monolith or of a single, commercial component. Also, the pump housing 19 can define a fluid inlet 27 and a pressurized fluid outlet 28. Fluid at an ambient pressure can be received by the pump 14 through the inlet 27, and the pump 14 can increase the pressure of the fluid before outputting the fluid through the outlet 28.

The sensor 22 can include a semiconductor pressure sensor or another type of pressure sensor. Additionally, other types of sensors, such as a temperature sensor, can also be included. The sensor 22 can be positioned within the pump housing 19 to detect an amount of air pressure in the hose 18. For example, the sensor 22 can be positioned in a portion of the pump 14 in communication with the hose 18, such as in fluid communication with the pressurized fluid outlet 28 of the pump 14 as shown in FIG. 1. Since the hose 18 can be in fluid communication with the bladder 16 of the mattress 12, the air pressure detected by the sensor 22 can indicate the air pressure in the bladder 16. While operation of the pump 14 may affect the pressure detected by the sensor 22, the pump 14 can operate only as required to maintain an average pressure within the bladder 16 (e.g., to replace any fluid that seeps out of the bladder 16). Additionally, the sensor 22 can draw power from a power source that also powers the pump 14, such as the electric line 21. The sensor 22 can output a pressure signal a to the controller 24. The sensor 22 can be hard-wired to the controller 24, the sensor 22 can wirelessly communication with the controller 24 by way of a transmitter using, for example, a standard wireless protocol (e.g., IEEE 802.11, RF, Bluetooth, or 3G), or the sensor 22 can otherwise be coupled to the controller 24 for communication therewith.

The controller 24, which can be a microprocessor or another device including a memory and a CPU for executing a program stored on the memory, can control a motor 26 in the pump 14 shown in FIG. 2 to produce pressurized air in the outlet 28 portion of the pump 14 shown in FIG. 1. The controller 24 can be hard-wired to the motor 26 or be in wireless communication with the motor 16 using, for example, a standard wireless protocol. As a result, the controller 24 can control the operation of the pump 14. For example, the controller 24 can control the pump 14 in response to the pressure signal a such as by instructing the pump 14 to inflate the bladder 16 when the controller 24 determines the air pressure in the bladder 16 is below a set amount. Thus, when the controller 24 actuates the motor 26, the motor 26 can produce pressurized air in the outlet 28 that passes from the pump 14 through the hose 18 and into the bladder 16 to increase the fluid pressure inside the bladder 16. The controller 24 can also be in communication with an air release valve or other structure for releasing air from the bladder 16 such that the controller 24 can provide an instruction to decrease the fluid pressure in the bladder 16. Also, while the controller 24 is shown as packaged with the pump 14, the controller 24 can alternatively be packaged with the control unit 15 or some other component besides the pump 14.

Additionally, the controller 24 can analyze the pressure signal a to determine a heart rate, respiration rate, and/or other vital signs of a user lying or sitting on the mattress 12. More specifically, when a user lies on the mattress 12, each of the user's heart beats, breaths, and other movements can create a force on the mattress 12 that is transmitted to the bladder 16. As a result of the force input to the bladder 16 from the user's movement, a wave can propagate through the bladder 16, into the hose 18, and arrive at the pump 14. The sensor 22 can detect the wave, and thus the pressure signal a output by the sensor 22 can indicate a heart rate, respiratory rate, or other information regarding user. If the pump 14 is of the type including a sensor 22 of the type originally designed for monitoring the fluid pressure within the bladder 16 to maintain the pressure at a substantially constant amount, a software upgrade can be used to increase the functionality of the pump 14 to determine the heart rate, respiratory rate, and other characteristics of the user without the need for a hardware modification. In this case, a hardware upgrade can provide the control unit 15, if desired.

To overcome a DC offset in the pressure signal α, the pressure signal α can pass through a circuit splitting the signal into a DC coupled path and an AC coupled path, and the AC coupled path can be amplified and filtered. The controller 24 can perform a pattern recognition algorithm or other calculation based on the amplified and filtered pressure signal a to determine the user's heart rate and respiratory rate. For example, the algorithm or calculation can be based on assumptions that a heart rate portion of the signal a has a frequency in the range of 0.5-4.0 Hz and that a respiration rate portion of the signal a has a frequency in the range of the range of less than 1 Hz. The controller 24 can also be configured to determine other characteristics of a user based on the pressure signal α, such as blood pressure, tossing and turning movements, rolling movements, limb movements, weight, the presence or lack or presence of a user, and/or the identity of the user. Further, the controller 24 can receive signals from other sensors (e.g., a temperature sensor). The controller 24 can output a status signal β indicating the characteristics of the user (e.g., heart rate and respiratory rate) to the control unit 15. Additionally, if multiple users are lying or sitting on the mattress 12, the pressure signal a detected by the sensor 22 can indicate each of the multiple users' vital signs, and the pattern recognition algorithm or other calculation performed by the controller 24 can detect each user's heart rate and respiration rate.

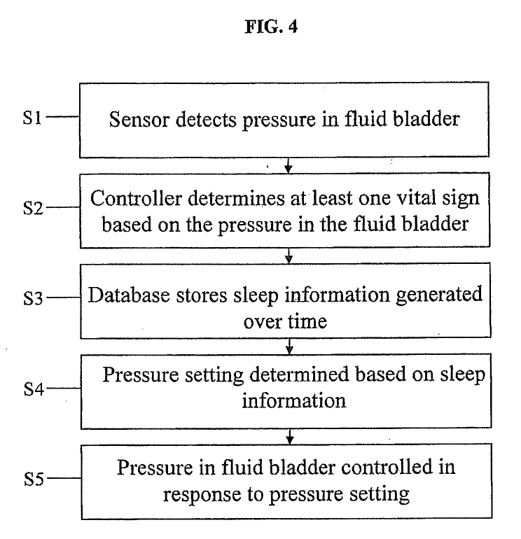

The control unit 15 can include a transmitter 30, a screen 32, and controls 34. The transmitter 30 can relay the status signal β to a database 36 or other source. The transmitter 30 can be a wireless transmitter operating using a standard wireless protocol (e.g., IEEE 802.11, RF, Bluetooth, or 3G) for communication with the database 36 or other source, though the transmitter 30 can alternatively be hardwired to the database using a phone line, Ethernet line, or other connection. As a result, the database 36 can store sleep information produced as a result of the status signal β, and the user can be alerted to sleep issues based on long-tern sleep trends or provided with other communications regarding the user's sleep (e.g., an alarm warning of apnea), fitness level, cardiovascular condition, or other health information. An example of storing sleep information with the database is discussed below in respect to FIG. 4.

The screen 32 can display information relayed in the status signal β, such as a sleep score based on the user's heart rate, respiratory rate, amount of time spend in REM sleep, total time in bed, and other considerations.

The control unit 15 can also be hard-wired or in wireless communication with the controller 24 for controller operation of the pump 14. As a result, the controls 34 can be used to control the operation of the sleep monitoring system 10. For example, the controls 34 can be used to increase the air pressure in the bladder 16. As another example, the controls 34 can be used to instruct the sensor 22 and/or controller 24 to operate in a privacy mode in which data is not detected, retained, displayed, transmitted, and/or analyzed, or to communicate with the database 36 to obtain sleep information (e.g., sleep trends, sleep scores from previous nights, sleeping tips). The database 36 can be accessible via the control unit 15 or a computer, e.g., via the internet.

As shown in FIG. 3, the bladder 16 can include multiple longitudinal supports 38 spaced across the bladder 16. The supports 38 can define channels 40 for fluid in the bladder 16 to pass from, for example, the head of the bladder 16 to the hose 18 via the inlet 17. That is, the supports 38 can be positioned not to impede waves propagating through the bladder 16 in a direction toward the sensor 24 (which in this case is through the inlet 17). The supports 38 can also provide support for a user lying on the mattress 12. A different arrangement of supports can be used, though the supports should not substantially hinder waves from propagating to the sensor 24. Also, the mattress 12 can include more than one bladder 16. For example, the mattress 12 can include two side-by-side bladders 16 for detecting the heart and respiratory rates of two users. In this case, the pump 14 can include more than one sensor 22.

The sleep monitoring system 10 can have a different structure from illustrated. For example, the pump 14 can include the transmitter 30 instead of the control unit 15. Additionally, the system 10 can have additional functions from those described above. For example, the control unit 15 can function as an alarm clock, and the alarm can be sounded until the system 10 determines that the user has awoken or got off the mattress 12.

As shown in FIG. 4, the sleep information stored in the database 36 can be used to improve the sleep of a user. In more detail, as shown in steps SI and S2 and discussed above in greater detail, the sensor 22 can detect the pressure in the fluid bladder 16 and the controller 24 can determine at least one vital sign based on the pressure in the fluid bladder 16.

As shown in step S3, the database 36 can store sleep information generated over time. The sleep information can include the pressure in the bladder 16, one or more vitals signs (e.g., heart rate, respiratory rate, etc.), a frequency or amount of tossing and turning by a user, a temperature, a light level, and other information. The sleep information need not necessarily include one of the vital signs, as one or more of the vital signs can be determined by a computer or other processing unit (i.e., a processor other than the controller 24). Also, the sleep information can be transferred to the database 36 by communicably linking the controller 24 and transmitter 30 and also communicably linking the transmitter 30 and database 36 as shown in FIG. 2, or in another way (e.g., directly communicably linking the sensor 22 and the database 36, or communicably linking the sensor 22 to the transmitter 30 and the transmitter 30 to the database 36).

The database 36 can store a log of sleep information as shown in step S3 of FIG. 4. For example, the database 36 can create a sleep score based on one or more vital signs. The sleep score can, for example, indicate high quality sleep when heart rate is low, when respiratory rate is low, and when tossing when turning movements are infrequent. Over time, the database 36 can accumulate sleep scores for a variety of conditions (e.g., a lower pressure in the bladder 16, a high pressure in the bladder 16, a cool temperature, a warm temperature, and/or a low level of light).

As shown in step S4, an association can then be made using the sleep information between the sleep score and environmental conditions, such as the pressure in the bladder 16, the light level, and the temperature. The association can be performed by the controller 24 or another processor in communication with the database 36. The association between the sleep score and environmental conditions can include, for example, determining a correlation between the sleep score and environmental conditions. Based on the association, a pressure setting can be determined for customizing the environmental conditions (e.g., pressure in the bladder 16, light level, and temperature) to achieve a high sleep score. Additionally, other settings (temperature and light level, for example) can be determined based on the association.

As shown in step S5, the controller 24 can control the pump 14 based on the pressure setting. For example, the controller 24 can actuate the motor 26 to inflate the bladder 16 if the pressure setting indicates a higher pressure would result in a higher sleep score. Further, other controls (e.g., a heater, air conditioner; and/or a night light) can be adjusted based on the association.

While the invention has been described in connection with what is presently considered to be the most practical example, it is to be understood that the invention is not to be limited to the disclosed example but, on tie contrary, is intended to cover various modifications and equivalent arrangements included within the spirit and scope of the appended claims, which scope is to be accorded the broadest interpretation so as to encompass all such modifications and equivalent structures as is permitted under the law.

1. A sleep monitoring system comprising:
a fluid bladder;
a pump in fluid communication with the fluid bladder, the pump operable to increase a fluid pressure within the fluid bladder;
a sensor packaged with the pump, the sensor in fluid communication with the fluid bladder and operative to determine a pressure within the fluid bladder; and
a controller configured to determine at least one vital sign based on the pressure within the fluid bladder.

2. The sleep monitoring system of claim 1, wherein the sensor is physically coupled to a housing containing components of the pump such that the sensor is part of an integral pump unit.

3. The sleep monitoring system of claim 1, further comprising an elongate conduit fluidly coupling the pump and the fluid bladder, and wherein the sensor is in fluid communication with the conduit.

4. The sleep monitoring system of claim 1, wherein the controller is operative to control the pump in response to the pressure within the fluid bladder.

5. The sleep monitoring system of claim 1, wherein the at least one vital sign includes at least one of a heart rate and a respiration rate.

6. The sleep monitoring system of claim 1, wherein the fluid bladder encloses pathways aligned with a fluid inlet.

7. The sleep monitoring system of claim 6, wherein an end of the fluid bladder defines the fluid inlet, and wherein the pathways extend longitudinally toward the fluid inlet.

8. (canceled)

9. The sleep monitoring system of claim 1, further comprising a control unit in communication with the controller, wherein the control unit includes a display configured to display the at least one vital sign.

10. The sleep monitoring system of claim 1, wherein the controller is operative to control the pressure within the fluid bladder in response to the at least one vital sign.

11. (canceled)

12. (canceled)

13. (canceled)

14. (canceled)

15. (canceled)

16. A sleep monitoring system comprising:
a fluid bladder;
a pump spaced from the fluid bladder, the pump having a housing containing pump components and defining a fluid inlet for receiving fluid and a fluid outlet for outputting fluid pressurized by the pump;
an elongate conduit fluidly coupling the fluid outlet of the pump and the fluid bladder, the conduit providing a passage for the fluid pressurized by the pump to increase a fluid pressure within the fluid bladder;
a pressure sensor disposed within the interior of the pump housing such that the sensor is part of an integral pump unit, the pressure sensor configured to detect a pressure of fluid at the fluid outlet of the pump; and
a controller coupled to the sensor and configured to determine at least one vital sign based on the pressure within the fluid bladder.

17. (canceled)

18. (canceled)

19. (canceled)

20. (canceled)

21. A mattress arrangement having the capability to monitor vital signs, comprising:
a bladder containing a fluid;
a pressure sensor in fluid communication with the fluid bladder and generating an output indicative of the pressure of the fluid; and
a pump in fluid communication with the fluid bladder, the pump responsive to the sensor output to increase fluid pressure when the pressure falls below a preset level;
a pump housing wherein the pump and pressure sensor is disposed within the interior of the pump housing such that the sensor and the pump comprise an integrated pump unit; and
a controller coupled to the sensor and configured to determine at least one vital sign of a person resting on the mattress based on the output of the pressure sensor.

22. The mattress arrangement of claim 21, wherein the controller is operative to change the preset level in response to the at least one vital sign.

23. The mattress arrangement of claim 21 wherein the controller is operative to control the pump in response to the output of the pressure sensor.

24. The mattress arrangement of claim 21 wherein the at least one vital sign includes at least one of a heart rate or a respiration rate.

25. The mattress arrangement of claim 21, wherein the fluid bladder encloses pathways aligned with a fluid inlet.

26. The mattress arrangement of claim 25, wherein an end of the fluid bladder defines the fluid inlet, and wherein the pathways extend longitudinally toward the fluid inlet.

27. The mattress arrangement of claim 25, wherein the fluid bladder includes parallel supports extending between a top side of the fluid bladder and a bottom side of the fluid bladder, the supports at least partially defining the pathways.

28. The mattress arrangement of claim 21, further comprising a control unit in communication with the controller, wherein the control unit includes a display configured to display the at least one vital sign.

29. The mattress arrangement of claim 21, further comprising a memory, wherein the controller is configured to store in the memory the value of the preset level and measurements of vital signs over time to provide an association of sleep quality and the preset level.

30. The mattress arrangement of claim 21, wherein the sensor and the pump comprise an integrated pump unit that is a monolithic commercial product sold separately from the controller configured to detect vital signs.
